(19) United States
(12) Patent Application Publication
(10) Pub. No.: US 2017/0368299 A1
(43) Pub. Date: Dec. 28, 2017

(54) SOUND SOURCE CONTROL DEVICE AND SOUND SOURCE CONTROL METHOD

(71) Applicant: YAMAHA CORPORATION, Hamamatsu-shi (JP)

(72) Inventors: Takehiko KAWAHARA, Hamamatsu-shi (JP); Kiyoshi YAMAKI, Iwata-shi (JP); Atsushi ISHIHARA, Iwata-shi (JP); Morito MORISHIMA, Fukuroi-shi (JP)

(21) Appl. No.: 15/678,297

(22) Filed: Aug. 16, 2017

Related U.S. Application Data
(63) Continuation of application No. PCT/JP2016/053745, filed on Feb. 9, 2016.

(30) Foreign Application Priority Data
Feb. 26, 2015 (JP) 2015-036870

(51) Int. Cl. A61M 21/02; A61B 5/0476; A61B 5/024; A61B 5/08; A61B 5/11; A61B 5/00; A61M 21/00

(52) U.S. Cl. CPC A61M 21/02; A61B 5/11; A61B 5/0476; A61B 5/4812; A61B 5/024; A61B 5/0816; A61M 2021/0027; A61M 2205/3303

(57) ABSTRACT

For the purpose of improving sleep satisfaction for different human subjects individually, a sound source control device (20) includes: an acquirer (202) configured to acquire a biorhythm of a human subject; an estimator (204) configured to estimate sleep depths of the human subject from the acquired biorhythm of the human subject; a controller (230) configured to control a sound source (240) to play a sound in accordance with the acquired biorhythm of the human subject, the sound being defined using a prescribed parameter set; and an evaluator (220) configured to evaluate a sleep state of the human subject based on the estimated sleep depths of the human subject, and based on the evaluation, instruct the controller (230) to change at least a part of contents of the parameter set.

### TECHNICAL FIELD

The present invention relates to a sound source control device and the like, which aims to improve sleep quality and the like by controlling a sound source.

### BACKGROUND ART

Recently, there have been proposed technologies for improving sleep of a human subject by detecting biorhythms of the human subject, such as body motion, respiration and heartbeat, and generating a sound in accordance with the detected biorhythms (for example, refer to Japanese Patent Application Laid-Open Publication No. H4-269972).

In addition, there have also been proposed technologies for adjusting, in accordance with a relaxation state of a human subject, at least one of a type, a volume, and a tempo of a generated sound (for example, refer to Japanese Patent Application Laid-Open Publication No. 2004-344284).

During sleep, rapid eye movement (REM) sleep, which is characterized by light sleep, and non-REM sleep, which is characterized by deep sleep, alternate in cycles of approximately 90 minutes. It is said a person awakens from REM sleep feeling relatively refreshed.

However, differences in cycles of REM sleep and non-REM sleep exist between individuals. Moreover, a surrounding environment or a period between sleep onset and awakening of a subject (human subject) may not always be the same.

### SUMMARY OF THE INVENTION

The present invention has been made in consideration of the aforementioned circumstances, and has as an object the provision of a technology by which sleep satisfaction can be improved for different human subjects individually.

To achieve the above-stated object, a sound source control device according to one aspect of the present invention has: a biorhythm acquirer configured to acquire a biorhythm of a human subject; an estimator configured to estimate sleep depths of the human subject from the acquired biorhythm of the human subject; a controller configured to control a sound source to play a sound in accordance with the acquired biorhythm of the human subject, the sound being defined using a prescribed parameter set; and an evaluator configured to evaluate a sleep state of the human subject based on the estimated sleep depths of the human subject, and based on the evaluation, instruct the controller to change at least a part of contents of the parameter set, wherein the evaluator evaluates the parameter set based on the sleep state of the human subject.

### BRIEF DESCRIPTION OF THE DRAWINGS

FIG. 1 illustrates an overall configuration of a system including a sound source control device according to an embodiment.

FIG. 2 is a block diagram showing a functional configuration of the system.

FIG. 3 is a table illustrating example control parameters.

FIG. 4 illustrates an example of sleep guidance.

FIG. 5A is a flowchart showing a sound source control process.

FIG. 5B is a flowchart showing a learning operation of the sound source control device.

FIG. 6 illustrates an example screen displayed on a display unit during the learning operation.

### MODES FOR CARRYING OUT THE INVENTION

In the following, embodiments of the present invention will be explained with reference to the drawings.

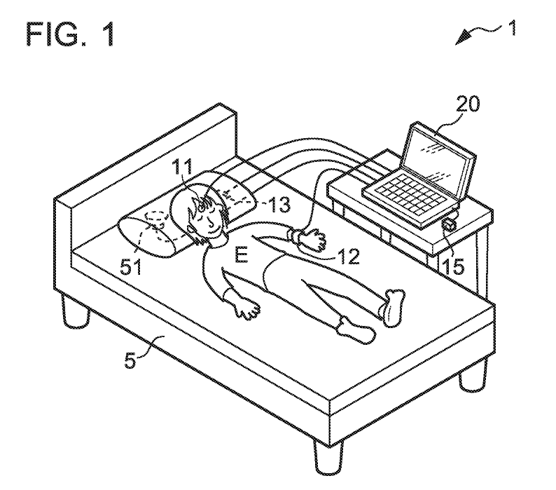

FIG. 1 illustrates an overall configuration of a system 1 including a sound source control device 20 according to an embodiment. As illustrated in the figure, the system 1 includes biosensors 11, 12, and 13, an environmental sensor 15, the sound source control device 20, and a loudspeaker 51.

The system 1 aims to improve sleep quality, for example, and in turn lead to enhanced sleep satisfaction by causing a sound output from the loudspeaker 51 to be heard (or perceived) by a human subject E lying on his/her back on a bed 5.

The forehead of the human subject E has attached thereto the biosensor 11, and the biosensor 11 detects brain waves (α wave, β wave, δ wave, θ wave, etc.) of the human subject E. The left wrist of the human subject E has attached thereto the biosensor 12, and the biosensor 12 detects pressure changes in the radial artery, i.e., a pulse wave, for example. Since a pulse wave is synchronous with a heartbeat, the biosensor 12 indirectly detects a heartbeat. The biosensor 13 is provided inside a pillow to detect pressure changes, acceleration, etc., relative to body motion of the human subject E, and thus the biosensor 13 detects respiration, heartbeat and so on from the detected pressure changes, acceleration, etc.

The biosensor 13 may be provided in a location other than the inside of the pillow, so long as the biosensor 13 can still detect biorhythms (respiration, heartbeat and so on) from body motion of the human subject E. For example, the biosensor 13 may be located between the pillow and the head of the human subject E, on the mattress, the sheet, etc., or inside the bed 5. The respiration, heartbeat, and so on may be detected indirectly instead, by way of reflected radio waves, sound waves, etc.

The environmental sensor 15 detects an environment around the human subject E. Specifically, the environmental sensor 15 detects a noise level, temperature and humidity, an air pressure, a luminance of ambient light, and so on.

Detected signals output from the biosensors 11, 12, and 13 and those output from the environmental sensor 15 are supplied to the sound source control device 20.

While the drawing illustrates a case where a single biosensor 11 is attached to the forehead of the human subject E, multiple ones of the biosensor 11 may be attached at different positions. For the sake of convenience, the drawing illustrates a configuration in which the detected signals of the biosensors 11, 12, and 13 are transmitted to the sound source control device 20 in wired form. However, wireless transmission may be employed instead. In a case where a heartbeat can be detected by the biosensor 13, the biosensor 12 may be omitted.

The sound source control device 20 mainly has the following functions: Namely, the sound source control device 20 processes detected signals output from the biosensors 11, 12, and 13 to estimate sleep depths of the human subject E; and in accordance with the estimated sleep depths, the sound source control device 20 controls playback of a sound that is caused to be heard by the human subject E such that the sound is linked with a biorhythm of the human subject E.

Here, “playing a sound such that the sound is linked with a biorhythm” basically means to play music at the same speed as (or at a speed close to) a cycle of a biorhythm, or to play a sound repeatedly at the same cycles as (or at cycles close to) those of a biorhythm, the sound being specified by waveform data. For example, a case is assumed where a cycle of biorhythm is presented in a form of heart rate. A heart rate is close to a general tempo of music. Thus, “playing a sound such that the sound is linked with a biorhythm” in this case means to play music at a tempo of a heart rate, or to repeatedly reproduce waveform data at cycles of the biorhythm. If a case is assumed where music is reproduced from MIDI data, a tempo of a biorhythm may be indicated in MIDI format.

“Playing a sound such that the sound is linked with a biorhythm” includes: playing music at a tempo that corresponds (or almost corresponds) to a cycle that is obtained by either dividing or multiplying a cycle of a biorhythm by an integer; and playing a sound at a cycle that corresponds (or almost corresponds) to a cycle that is obtained by either dividing or multiplying a cycle of a biorhythm by an integer.

In the above explanation, an example is given where control of tempo and a related process are performed. When volume is a subject of control, the control also causes a sound to fade in at the beginning of a cycle of a biorhythm (volume is caused to increase from zero), and causes a sound to fade out at the end of a cycle of a biorhythm (volume is caused to decrease to zero).

As stated above, “playing a sound such that the sound is linked with a biorhythm” means manipulating a tempo, an amplitude, etc., of the sound in accordance with the biorhythm.

The sound source control device 20 may be a portable terminal or a personal computer, for example. Functional blocks (described later) are realized by a central processing unit (CPU) of the sound source control device 20 executing a computer program installed in advance. In the example illustrated in the drawing, the sound source control device 20 is illustrated as a personal computer, but the sound source control device 20 may instead be provided inside the pillow, for example.

In this example, the loudspeaker 51 is provided inside the pillow, and causes a sound output from the sound source control device 20 to be heard by the human subject E. An alternative configuration may employ headphones to cause a sound to be heard by the human subject E. However, the present embodiment is explained based on a configuration in which the loudspeaker 51 is used.

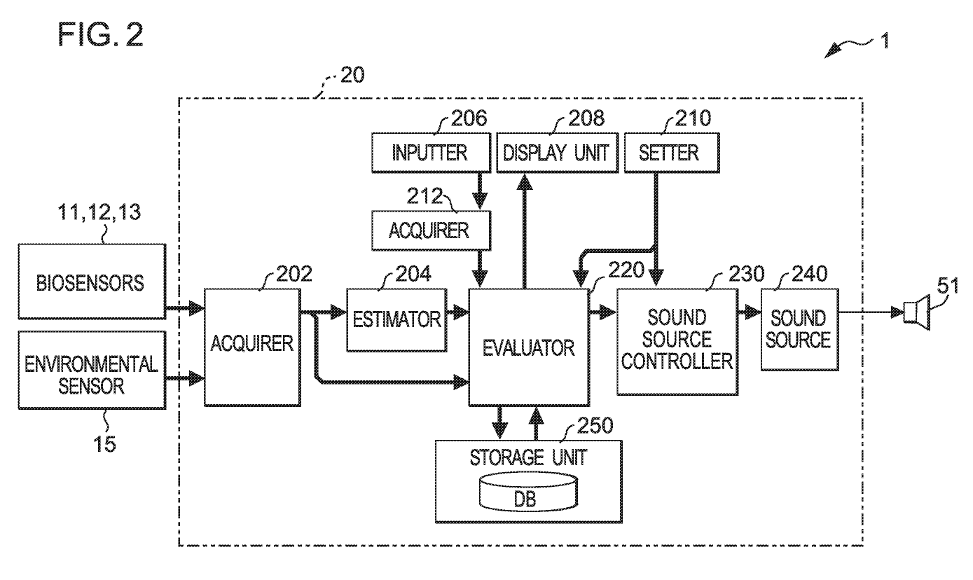

FIG. 2 mainly illustrates a configuration of functional blocks in the sound source control device 20 of the system 1. As illustrated in this figure, the sound source control device 20 includes acquirers 202 and 212, an estimator 204, an inputter 206, a display unit 208, a setter 210, an evaluator 220, a sound source controller 230, a sound source 240, and a storage unit 250. These functional blocks are realized by the CPU (not shown) executing the aforementioned computer program.

The acquirer 202 in FIG. 2 has an internal memory. The acquirer 202 converts the detected signals of the biosensors 11, 12, and 13 into digital signals and temporarily accumulates the same in the internal memory, and then supplies the signals to the estimator 204 and the evaluator 220.

Based on the detected signals of the biosensors 11, 12, and 13, the estimator 204 estimates whether a current sleep depth of the human subject E corresponds to non-REM sleep, REM sleep, or wakefulness. In estimating sleep depths in the present embodiment, non-REM sleep is divided into four stages 4 to 1 in decreasing order of depth of sleep. For the sake of convenience, a total of six stages are used for estimating sleep depths. However, time changes (characteristics) (described later) of estimated sleep depths are shown in the drawings without being defined in stages.

An approach such as the following may be employed to estimate sleep depths. Specifically, a calm state in which there is relatively little body motion yet a β wave is dominant is defined as “wakefulness”, whereas a state in which a θ wave is present yet respiration is shallow and irregular is defined as “REM-sleep”. In non-REM sleep, a state of light non-REM sleep where a θ wave is present is defined as “stage 1”; and a state of deep non-REM sleep where a δ wave is present is defined as “stage 4”. Between “stage 1” and “stage 4” there are two additional stages, which in order from “stage 1” are “stage 2” and “stage 3”.

The evaluator 220 mainly has the following functions.

Firstly, from among multiple sets of control parameters stored in a database DB of the storage unit 250, the evaluator 220 selects and reads a set of control parameters for use in sleep guidance, and transmits the same to the sound source controller 230.

The control parameters include multiple parameters, such as a type of sound source, tempo control, and volume control, as will be described later. For this reason, a group consisting of multiple parameters is expressed as a “set”.

Secondly, the evaluator 220 indicates control contents to the sound source controller 230, so that sleep depths of the human subject E estimated by the estimator 204 follow target characteristics (described later) of sleep depths. In so doing, the evaluator 220 periodically transmits to the sound source controller 230 biorhythms (α respiratory rate, or the like) detected by the biosensors 11, 12, and 13.

Thirdly, following completion of a sleep period of the human subject E, the evaluator 220 writes an evaluation result of the sleep period with regard to a control parameter in the database DB, which is used for the latest period of sleep. Alternatively, the evaluator 220 writes, with regard to the control parameter, a result by adding an evaluation result of the latest sleep to an evaluation result of a prior sleep that has already been written. In this manner, a relationship between a prior sleep period of the human subject E and each control parameter can be quantified.

Fourthly, the evaluator 220 causes the database DB in the storage unit 250 to store characteristics indicating a manner in which sleep depths estimated for the latest sleep period of the human subject E changed over time.

The storage unit 250 includes the database DB, and stores sets of control parameters and averaged characteristics (described later) of sleep depths of the human subject E.

The inputter 206 is used by the human subject E to input a result of his/her subjective evaluation of a sleep, following completion of the sleep period. A result of this input is supplied to the evaluator 220 via the acquirer 212. The display unit 208 is configured to display various information items and evaluation results by the evaluator 220. The setter 210 is used to set an awakening time, and to input various conditions and instructions. The inputter 206, the display unit 208, and the setter 210 may be integrated into a single functional block in a form of, for example, a liquid-crystal display panel provided with a touchscreen.

The sound source controller 230 controls the sound source 240 in accordance with the control contents indicated by the evaluator 220 and the control parameter received from the evaluator 220.

In accordance with the control performed by the sound source controller 230, the sound source 240 generates (reproduces) a sound in a form of a digital signal. The generated signal is converted into an analog signal, and is then output as sound from the loudspeaker 51, to be heard by the human subject E.

The sound source 240 in this example has been described in terms of a so-called software sound source realized by executing a computer program, but needless to state, the sound source 240 may also be realized in a form of a hardware sound source.

Although differences exist between individuals, non-REM sleep and REM sleep generally repeat alternately during human sleep, and it is said that a person is likely to feel refreshed when awaking from REM sleep during this alternating cycle.

Accordingly, in the present embodiment, the sleep depth of the human subject E is guided such that the sleep depth is REM-sleep at an awakening time Ti.

A person is more likely to relax and fall asleep, with sleep becoming deeper, when hearing a sound at cycles that are longer than cycles of biorhythms, such as heartbeat, respiration, and brain waves. In particular, a person is more likely to fall asleep when hearing hypersonic sound at a frequency of 20 kHz or above.

In contrast, when hearing a sound at cycles that are shorter than cycles of biorhythms, a person is more likely to become excited, and the sleep period is more likely to transition toward wakefulness (sleep becomes lighter).

In consideration of the above, the sound source control device 20 is configured to either lengthen or shorten a cycle of a sound to be heard by the human subject E, relative to a cycle of a biorhythm (e.g., respiration) detected by the biosensors 11, 12, and 13, so as to appropriately guide a sleep depth. In this way, the sound source control device 20 promotes an improvement in sleep quality, and guides a sleep depth such that the sleep depth at the awakening time corresponds to REM sleep. In this case, control for guiding a sleep depth is performed by referring to the following control parameters.

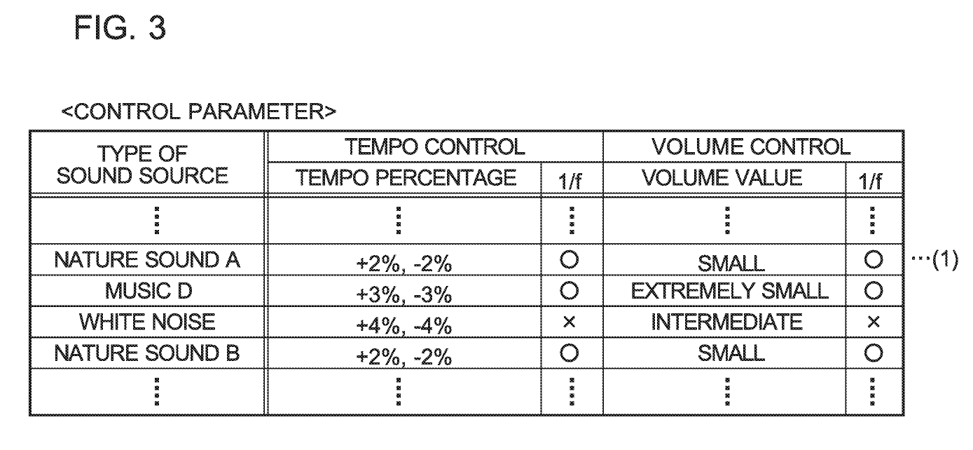

FIG. 3 illustrates example control parameters stored in the storage unit 250. In the control parameters illustrated in this figure, contents of tempo control and volume control are specified for each type of sound source.

The expression “type of sound source” refers to a type of sound played to improve sleep. In the figure, nature sound A, music D, white noise, and nature sound B are given as examples of such sounds that can be played. Various kinds of soothing nature sounds exist, such as a sound of gentle waves and a sound of wind. In the figure, different nature sounds are distinguished by being denoted by signs “A”, “B”, etc., respectively. Signs denoting different forms of music serve the same purpose.

The tempo control has two categories, namely tempo percentage and 1/f.

The expression “tempo percentage” specifies a degree (percentage) by which a cycle of a sound to be heard by a human subject is lengthened or shortened relative to a cycle of a biorhythm. An example will now be given of a case where the type of sound source corresponds to “nature sound A” in FIG. 3. The tempo percentage indicates a tempo for a sound that is slower than the cycle of the biorhythm by 2 percent (minus 2%) when exposing the human subject to a sound having a longer cycle than the cycle of the biorhythm. On the contrary, the tempo percentage indicates a tempo for a sound that is faster than the cycle of the biorhythm by 2 percent (additional 2%) when exposing the human subject to a sound having a shorter cycle than the cycle of the biorhythm.

When the tempo of the sound remains constant under control with this tempo percentage, the sound tends to become monotonous. In this regard, “1/f” is used to specify whether or not 1/f fluctuation should be introduced to a cycle of a sound specified using the tempo percentage. Specifically, with “o” (circle) it is specified that such fluctuation is to be introduced, and with “x” it is specified that such fluctuation is not to be introduced.

The volume control has two categories, namely a volume value and 1/f.

“Volume value” is used to specify a volume of a sound output from the loudspeaker 51. When a specified volume is uniform, the sound again tends to become monotonous. In this regard, “1/f” is used to specify whether or not 1/f fluctuation should be introduced to a volume specified using the “volume value”. Specifically, with “o” it is specified that such fluctuation is to be introduced, and with “x” it is specified that such fluctuation is not to be introduced.

Moreover, although not shown in FIG. 3, the control parameters are provided with items (fields) into which evaluation results of the evaluator 220 evaluating a sleep period are written. If is of note that FIG. 3 is merely one example. A greater number of control items may be provided to make control more complex, or multiple types of sound sources may be combined for use. For example, each control parameter may be provided with an item “band”, which specifies a frequency band that is either emphasized or deemphasized by an additionally-provided equalizer.

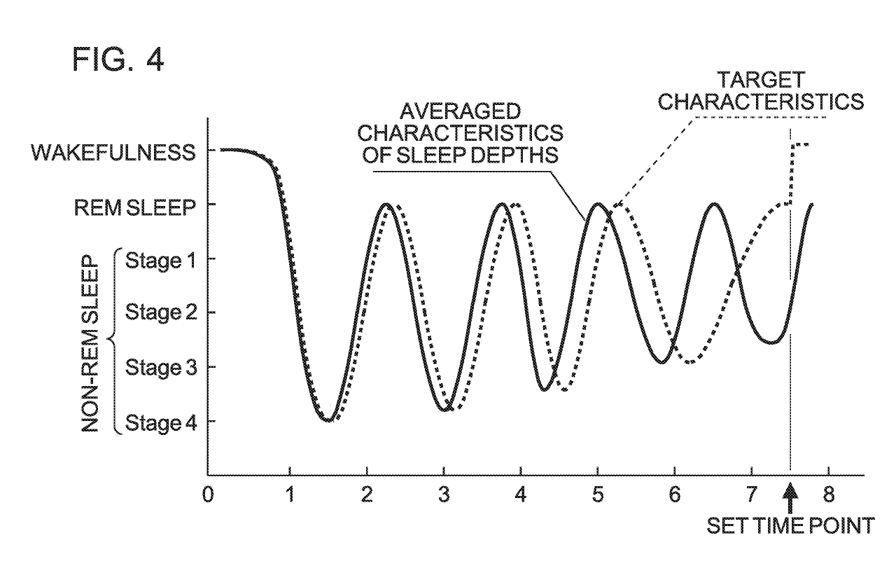

FIG. 4 illustrates an example of characteristics of sleep depths of the human subject E estimated by the sound source control device 20. The solid line in the figure is indicative of averaged characteristics of sleep depths of the human subject E. The “averaged characteristics of sleep depths” are averages of characteristics indicative of time changes in sleep depths of multiple sleep periods, which were estimated under conditions where sleep depths were not guided. The averaged characteristics may be obtained, for example, by estimating and recording sleep depths over time for a single sleep period extending from sleep onset to awakening, and from among characteristics of sleep depths estimated and recorded for multiple sleep periods, averaging characteristics of sleep depths of sleep periods for which favorable evaluations were obtained after respective sleep periods in terms of sleep satisfaction of the human subject.

The averaged characteristics of sleep depths thus obtained are stored in the database DB of the storage unit 250.

During sleep, as indicated by the solid line, non-REM sleep and REM sleep generally alternate with each other, and the deepest values of sleep depths tend to show a gradual rise over time (i.e., the sleep depth becomes gradually lighter). A “deepest value” as referred to herein is a value corresponding to a deepest point that a sleep depth reaches in a transitional course of REM sleep→non-REM sleep→REM sleep. It is of note that a deepest value may not only be a local minimum obtained at a point where a fall turns into a rise, but may also be a horizontal value corresponding to a value that remains constant (horizontal) between a fall and a rise.

Next, an operation of the sound source control device 20 will be explained.

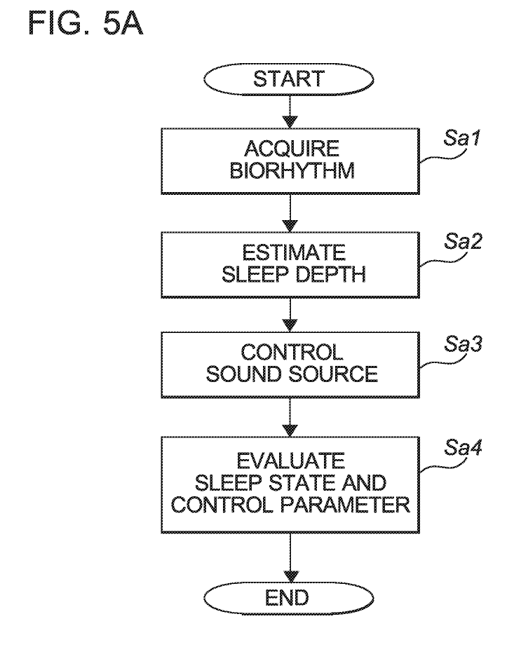

FIG. 5A is a flowchart showing an outline of sound source control performed by the CPU of the sound source control device 20 in sleep guidance.

As shown in the figure, the CPU acquires biorhythms of the human subject E (body motion, respiration, a heartbeat, etc.) from the biosensors 11, 12, and 13 (step Sa1), and estimates a sleep depth of the human subject E from the acquired biorhythms of the human subject E (step Sa2). Next, the CPU controls the sound source 240 to play a sound defined by the selected control parameters, in accordance with the estimated sleep depth and in such a way that the sound is linked with the acquired biorhythms of the human subject (step Sa3). The CPU repeatedly performs the operation from steps Sa1 to Sa3 at prescribed intervals from sleep onset to awakening of the human subject. After the human subject awakes, the CPU evaluates a sleep state of the human subject based on target characteristics and characteristics of the estimated sleep depths of the human subject and further, based on the sleep state of the human subject, evaluates the control parameters used. The CPU, based on this evaluation, changes at least a part of the contents of the control parameters (step Sa4; the learning operation (described later)).

Details of a sleep guidance operation by way of sound source control will now be explained. Description is given below on the assumption that, from among the control parameter sets in FIG. 3, the control parameter set (1) is selected by the human subject and is used for generating a sound.

First, when the human subject inputs an awakening time to the setter 210, the evaluator 220 acquires information indicating the set awakening time. The evaluator 220 reads, from the database DB of the storage unit 250, the averaged characteristics of the sleep depths of the human subject E (the solid line in FIG. 4) and the control parameter set (1), and supplies the control parameter set (control parameters) to the sound source controller 230.

The sound source controller 230 controls the sound source 240 to generate, for example, a hypersonic sound that is suitable for guiding the human subject E to fall asleep. Consequently, the loudspeaker 51 outputs the hypersonic sound, and the human subject E is guided to fall asleep.

The evaluator 220 determines that the human subject E has fallen asleep in a case in which the sleep depth estimated from the detection results of the biosensors 11, 12, and 13 has become deeper than a threshold depth (e.g., a depth that is slightly lighter than REM sleep), and indicates to the sound source controller 230 control contents indicating that the hypersonic sound should be switched to a sound specified by the supplied control parameter set. In accordance with the control contents, the sound source controller 230 controls the sound source 240.

Since the control parameter set (1) is used as described above, the loudspeaker 51 outputs nature sound A, which is controlled to accord with the acquired biorhythm (tempo) with 1/f fluctuations, and to have a low volume with 1/f fluctuations.

Regarding tempo control, it is of note that based on the control contents indicated by the evaluator 220 to the sound source controller 230, the expression tempo control is used to mean controlling a tempo of the sound to be slower by 2% (i.e., minus 2%) relative to a biorhythm (tempo) in the case of guiding a sleep depth toward a deeper state; and controlling a tempo of the sound to be faster by 2% (additional 2%) relative to the biorhythm (tempo) in the case of guiding a sleep depth toward a lighter state; and controlling the sound with 1/f fluctuations (details will be described later).

The evaluator 220 modifies the averaged characteristics of the sleep depths (characteristics indicated by the solid line in FIG. 4) for use as target characteristics (characteristics indicated by the broken line in FIG. 4) in sleep guidance, where the averaged characteristics of the sleep depths are modified by either being extended or contracted along a time axis such that the sleep depth at an awakening time corresponds to REM sleep.

Specifically, the evaluator 220 first determines a set awakening time as a set time point, by taking account of a time that elapses from a start of sleep to the set awakening time. For example, when a time point at which a human subject is determined to have fallen asleep is 11 pm and a set awakening time is 6:30 am the following day, the evaluator 220 obtains as a set time point a time point at which 7 hours and 30 minutes will have elapsed from the start point of the sleep depths (the time point at which the human subject is determined to have fallen asleep) as indicated by the solid line.

Secondly, the evaluator 220 modifies the averaged characteristics of the sleep depths indicated by the solid line by either extending or contracting the same so that REM sleep exists at the set time point. In other words, the evaluator 220 modifies the averaged characteristics of the sleep depths by either delaying phases of the characteristics gradually when extending the characteristics along the time axis, or by advancing the phases gradually when contracting the characteristics along the time axis, so that it will be REM sleep at the set time point.

The example broken line in FIG. 4 illustrates a case where the averaged characteristics of the sleep depths are extended along the time axis (the phases are gradually delayed), so that REM sleep exists at the set time point.

Thirdly, the evaluator 220 estimates a current sleep depth by the estimator 204 from the biorhythms detected by the biosensors 11, 12, and 13, while recording the estimated sleep depth. The evaluator 220 then compares the estimated sleep depth with the target characteristics obtained by modifying the averaged characteristics, to indicate to the sound source controller 230 control contents such as the following.

Specifically, when the estimated sleep depth is deeper than the sleep depth of the target characteristics where the estimated sleep depths are changing toward a deeper sleep state, the evaluator 220 indicates control contents to the sound source controller 230, according to which control contents a sound having a shorter cycle than the cycle of the biorhythm is generated. Accordingly, the sound source controller 230 causes the sound source 240 to accelerate a tempo of playback of the nature sound A to be faster than the biorhythm by 2 percent, and thus, a transition speed of sleep depths of the human subject E changing toward a deeper sleep state is directed toward a decrease (so that deepening of the sleep depth of the human subject becomes slower). In this way, the sleep depths are caused to approach the target characteristics.

On the other hand, when the estimated sleep depth is lighter than the sleep depth of the target characteristics where the estimated sleep depths are changing toward a deeper sleep state, the evaluator 220 indicates control contents to the sound source controller 230, according to which control contents a sound having a longer cycle than the cycle of the biorhythm is generated. Accordingly, the sound source controller 230 causes the sound source 240 to decelerate a tempo of playback of the nature sound A to be slower than the biorhythm by 2 percent, and thus, a transition speed of sleep depths of the human subject E changing toward a deeper sleep state is directed toward an increase (so that deepening of the sleep depth of the human subject becomes faster). In this way, the sleep depths are caused to approach the target characteristics.

When the estimated sleep depth is deeper than the sleep depth of the target characteristics where the estimated sleep depths are changing toward a lighter sleep state, the evaluator 220 indicates control contents to the sound source controller 230, according to which control contents a sound having a shorter cycle than the cycle of the biorhythm is generated. Accordingly, the sound source controller 230 causes the sound source 240 to accelerate a tempo of playback of the nature sound A to be faster than the biorhythm by 2 percent, and thus, a transition speed of sleep depths of the human subject E changing toward a lighter sleep state is directed toward an increase. In this way, the sleep depths are caused to approach the target characteristics.

On the other hand, when the estimated sleep depth is lighter than the sleep depth of the target characteristics where the estimated sleep depths are changing toward a lighter sleep state, the evaluator 220 indicates control contents to the sound source controller 230, according to which control contents a sound having a longer cycle than the cycle of the biorhythm is generated. Accordingly, the sound source controller 230 causes the sound source 240 to decelerate a tempo of playback of the nature sound A to be slower than the biorhythm by 2 percent, and thus, a transition speed of sleep depths of the human subject E changing toward a lighter sleep state is directed toward a decrease. In this way, the sleep depths are caused to approach the target characteristics.

As a result of such control, the sleep depths estimated for the human subject E change almost in exact correspondence with the sleep depths of the target characteristics, and therefore, REM sleep will exist at the set time point and the human subject E will awaken feeling refreshed.

At the set time point, the sound source controller 230 causes an alarm to sound, accelerates a tempo of the sound, increases a volume, or uses other such means to guide the human subject to awakening.

Differences between individuals are not the only factors that influence (result in changes in) sleep satisfaction. For example, even when characteristics of sleep depths estimated in a single sleep period change in the same manner as the averaged characteristics of sleep depths, a factor, such as a difference in health condition, may influence an overall satisfaction that a person obtains from sleep. Moreover, in association with a personal taste of a human subject, contents of tempo control, contents of volume control, and a type of sound source specifying a sound to be heard by the human subject during sleep, may influence sleep satisfaction.

In this regard, a learning operation will next be described in which, for the purpose of further improving sleep satisfaction, evaluation results of a sleep are reflected in a control parameter used in sleep guidance, with the control parameter being evaluated to learn an influence that the sleep guidance using the control parameter exerts on sleep satisfaction.

FIG. 5B is a flowchart showing a learning operation for learning a control parameter.

This learning operation is executed upon occurrence of a prescribed event, such as the human subject E terminating sleep guidance after the awakening time.

The evaluator 220 first evaluates a sleep period that immediately precedes the event (step Sb11).

Here, evaluation of sleep is categorized into objective and subjective ones. Objective evaluation is that which is free from subjectivity of the human subject. Subjects of objective evaluation may include at least one of: estimated sleep depths; quantified stability of sleep cycles corresponding to time intervals between REM sleep and non-REM sleep obtained from estimated sleep depths; and a degree of similarity between estimated sleep depths and target characteristics or between sleep cycles and target characteristics. Moreover, subjects of objective evaluation may also include one of the following: a time it takes from a time point at which a prescribed operation is performed on the setter 210 to a time point at which sleep onset of the human subject is determined; a period it takes from a time point at which an alarm alerting arrival of the awakening time sounds to a time point at which a sleep depth is estimated to have reached wakefulness; and when a snooze function is used, a quantified number of times the alarm sounds, for example. The “snooze function” causes an alarm to once again sound after a prescribed period of time has elapsed since the alarm was turned off.

A configuration may be employed in which, if a sleep environment is determined to correspond to an exceptional condition by referring to a temperature, humidity, and/or light-intensity detected by the environmental sensor 15, evaluation results obtained in that sleep environment may either be compensated for or excluded from the learning operation.

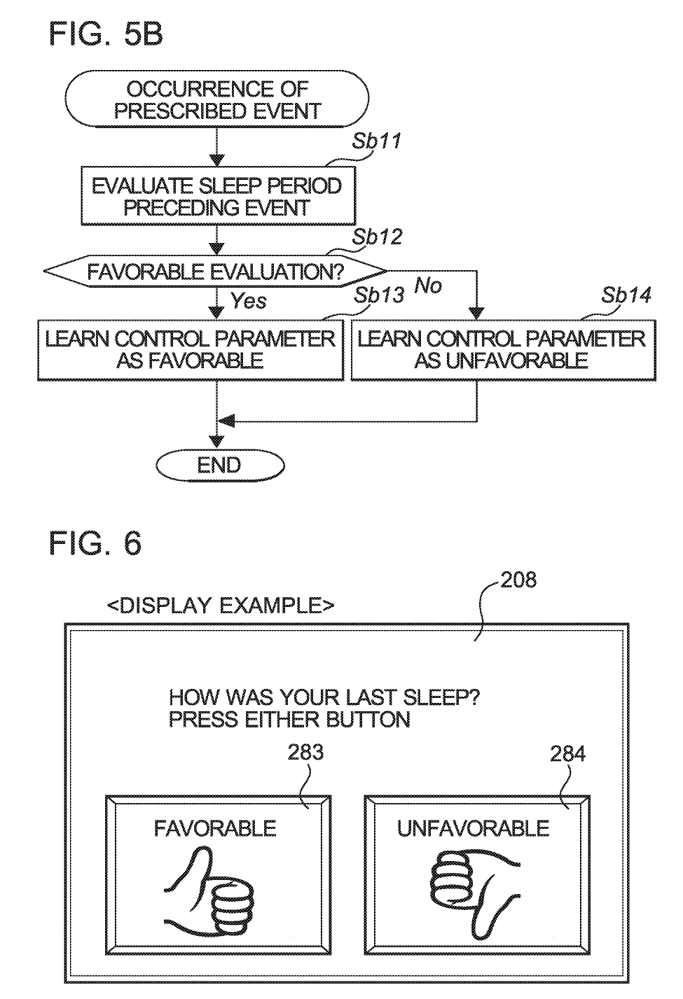

Subjective evaluation refers to evaluation that is based solely on a subjective impression of the human subject. To conduct such subjective evaluation, the evaluator 220 may for instance cause the display unit 208 to display a screen as shown in FIG. 6. In the example screen in FIG. 6, the display unit 208 is caused to display a software button 283 for receiving an input indicating favorable evaluation of sleep satisfaction, and a software button 284 for receiving an input indicating unfavorable evaluation of sleep satisfaction, with the human subject being prompted to operate either one of the buttons.

In this example, a choice is made between two alternatives (favorable or unfavorable). However, an alternative configuration may be employed in which a choice is made from among three or more levels of evaluation, or in which the number of points indicative of a degree of satisfaction is directly input, or in which only a favorable evaluation is input. Alternatively, a configuration may be employed in which a choice includes only an unfavorable evaluation. The parameters that are evaluated as unfavorable may be excluded from use in subsequent sleep periods.

The evaluator 220 adds up the thus obtained objective and subjective evaluation results, each of which is weighted in accordance with a predetermined proportion, and based on whether or not an addition result is equal to or more than a threshold, the evaluator 220 determines whether the evaluation of the sleep period before the aforementioned event was favorable or unfavorable (step Sb12).

If the evaluation is favorable (if the determination result in step Sb12 is “Yes”), the evaluator 220 learns that the control parameter set used in the evaluated sleep is a favorable control parameter set (step Sb13). For example, a configuration may be employed in which the evaluator 220 increases a priority of the control parameter set (1) used, thereby raising a position that the control parameter set (1) occupies in candidate rankings of control parameter sets to be selected for subsequent sleep guidance.

If the evaluation is unfavorable (if the determination result in step Sb12 is “No”), the evaluator 220 learns that the control parameter set used in the evaluated sleep is an unfavorable control parameter set (step Sb14). For example, a configuration may be employed in which the evaluator 220 decreases a priority of the control parameter set (1) used, thereby lowering a position that the control parameter set (1) occupies in candidate rankings of control parameter sets to be selected for subsequent sleep guidance.

The learning operation is terminated after step Sb13 or step Sb14.

As a result of such a learning operation, selection of a suitable control parameter for the human subject is facilitated, and hence, improvement of sleep satisfaction is also facilitated.

Once such evaluation of a control parameter set used in an evaluated sleep period is completed, in a subsequent sleep period the evaluator 220 selects a control parameter set occupying a high candidate ranking from among multiple control parameter sets. Then, in substantially the same manner as described above, the selected control parameter set is used in subsequent sleep guidance performed on the human subject, and the evaluator 220 evaluates the control parameter set used in the sleep guidance. In an alternative embodiment, all available control parameter sets may be evaluated first, after which a control parameter set having a high ranking may be selected from among the thus evaluated control parameter sets.

It is of note that a configuration may be employed in which target characteristics used in sleep guidance are apportioned into time segments in accordance with stages of sleep depths; a control parameter set is specified uniquely for a sleep period in each segment; and the evaluator 220 individually evaluates control parameter items (type of sound source, tempo control, volume control, etc.). In other words, by specifying the same control parameter set for the respective segments and evaluating the control parameter set with respect to each segment, it is possible to determine whether the use of the subject control parameter set is effective in lighter sleep stages or deeper sleep stages. Alternatively, different control parameter sets may be specified for respective segments and thus evaluated.

Thus, a configuration may be employed in which the sound source 240 is controlled using a different control parameter set for each sleep cycle by apportioning the target characteristics used in sleep guidance into time segments each corresponding to one sleep cycle; and in which the evaluator 220 evaluates each of the different control parameter sets based on a sleep state of the human subject in each sleep cycle.

Furthermore, a new control parameter set may be created by combining items that have received favorable evaluation. For example, the following approach may be employed in creating a control parameter set used for deep sleep. For a time segment corresponding to a deep sleep stage that is reached following sleep onset, sleep is guided so as to remain deep for a given period, by using a control parameter set A in which the type of sound source is set to nature sound A, the tempo percentage is set to ±2%, and the volume value is set to be low. Then, these items are evaluated. Thereafter, for a time segment corresponding to a deep sleep stage in the following sleep cycle, sleep is guided so as to remain deep for a given period, by using a different control parameter set, which is a control parameter set B, in which the type of sound source is set to music D, the tempo percentage is set to ±3%, and the volume value is set to be extremely low. Then, these items are evaluated. The guidance and evaluation are performed repeatedly for multiple segments, and from among items in the control parameter sets A and B, those items that have received a high evaluation are combined to create a control parameter set used for deep sleep. In a case where both control parameter sets A and B have received a high evaluation, two separate channels of the sound source 240 may be used to simultaneously play two sounds that are defined using the control parameter sets A and B, respectively.

An alternative configuration may be employed in which items that are likely to be useful are combined, as appropriate, and are used as a control parameter set to be evaluated and learned. In other words, in a case where a large number of evaluations are taken into account, if setting the type of sound source to nature sound D tends to often result in a high evaluation regardless of a tempo and a volume, and setting the tempo percentage to ±3% tends to often result in a high evaluation regardless of a type of sound source and a volume, then a control parameter set may be newly created that consists of a combination of the nature sound D and the tempo percentage of ±3%.

Biorhythms may be acquired in a form of data that is detected by (a) similar external device(s) to the biosensors 11, 12, and 13, instead of being detected by the biosensors 11, 12, and 13. Likewise, subjective evaluation of sleep may be acquired in a form of data input by a device differing from the inputter 206, instead of being input by an operation performed on the inputter 206.

Moreover, a stimulus other than a sound may be used in sleep guidance, such as fragrance (olfactory stimulus), ambient light (visual stimulus), temperature, humidity, and vibration (tactile stimulus). Two or more of the above stimuli including sound (auditory stimulus) may be used in an appropriate combination for sleep guidance.

The aforementioned computer program may be provided by being stored in a computer-readable recording medium for installation in a computer. For instance, the storage medium may be a non-transitory storage medium, a favorable example of which is an optical storage medium, such as a CD-ROM (optical disc), and may also be a freely-selected form of well-known storage media, such as a semiconductor storage medium and a magnetic storage medium. The computer program of the present invention may be provided by being distributed via a communication network for installation in a computer.

The following aspects of the present invention may be derived from the different embodiments and modifications described in the foregoing.

A sound source control device according to one aspect of the present invention has: a biorhythm acquirer configured to acquire a biorhythm of a human subject; an estimator configured to estimate sleep depths of the human subject from the acquired biorhythm of the human subject; a controller configured to control a sound source to play a sound in accordance with the acquired biorhythm of the human subject, the sound being defined using a prescribed parameter set; and an evaluator configured to evaluate a sleep state of the human subject based on the estimated sleep depths of the human subject, and based on the evaluation, instruct the controller to change at least a part of contents of the parameter set, wherein the evaluator evaluates the parameter set based on the sleep state of the human subject.

According to the sound source control device in the aforementioned aspect, a sleep state of the human subject is evaluated by the estimator, and a part of a prescribed parameter set is changed based on a result of the evaluation. Thus, sleep satisfaction can be improved for different human subjects individually, without a need for a human subject or the like to adjust the parameter set.

The sound source control device according to the aforementioned aspect may be configured such that in accordance with a set time point, the evaluator modifies characteristics indicative of time changes in sleep depths that are stored in advance in association with the human subject, and the evaluator instructs the controller to change the at least a part of the contents of the parameter set such that characteristics indicative of time changes in the estimated sleep depths of the human subject approach the modified characteristics. According to this configuration, a sleep depth of a human subject can be guided so as to be light at a set time point, and therefore, an improvement in sleep satisfaction can be expected.

Preferably, the evaluator apportions the modified characteristics into time segments; transmits to the controller the parameter set for each of the time segments; instructs the controller to change the at least a part of the contents of the parameter set such that, for each of the time segments, the characteristics indicative of the time changes in the estimated sleep depths of the human subject for that segment approach the modified characteristics in that segment; and evaluates the parameter set based on a sleep state of the human subject in each of the time segments.

Moreover, the evaluator may transmit to the controller the parameter set to each time segment, the parameter set being one of parameter sets different from one another for respective ones of the time segments, and may evaluate each of the parameter sets based on a sleep state of the human subject in a corresponding one of the time segments.

The sound source control device according to the aforementioned aspect may be configured such that the evaluator evaluates the prescribed parameter set based on an evaluation result of the sleep, the evaluation being performed by the human subject.

In the sound source control device according to the aforementioned aspect, evaluation of a sleep state may be an objective evaluation that is independent from any subjective evaluation by the human subject. Examples of evaluation items used in such objective evaluation include cycles of estimated sleep depths, stability of such cycles, and a time (period) it takes for the human subject to fall asleep or awaken. Examples of conditions setted in the prescribed parameter set include a type of sound, a volume, and a tempo percentage.

Evaluation conducted by the human subject may be subjective. Subjective evaluations may be of two types, namely, favorable or unfavorable; or may be of one type, namely, favorable. Alternatively, three or more evaluations may be employed step-wise. These subjective and objective evaluations of sleep may be converted into points, weighted, and added up. If a result of the addition is equal to or more than a threshold, a control parameter set used may be evaluated as favorable, whereas if the result of the addition is less than the threshold, the control parameter set may be evaluated as unfavorable.

Furthermore, a display unit may be provided to display subjective evaluations, objective evaluations, a device setting status, etc.

An environmental sensor may be provided to detect at least one of a surrounding noise level, ambient (room) temperature, humidity, a luminance and an air pressure. Detection results by the environmental sensor may be reflected in a parameter set or be displayed on the display unit mentioned above.

The present invention may be embodied not only in a form of a sound source control device, but also in a form of a sound source control method, or a program that causes a computer to function as the sound source control device, or a computer-readable recording medium on which the program is stored.

### DESCRIPTION OF REFERENCE SIGNS

11, 12, 13: biosensors
15: environmental sensor
20: sound source control device
202: acquirer (biorhythm acquirer)
204: estimator
212: acquirer
220: evaluator
230: sound source controller (controller)
240: sound source
250: storage unit
51: loudspeaker

1. A sound source control device comprising:
a biorhythm acquirer configured to acquire a biorhythm of a human subject;
an estimator configured to estimate sleep depths of the human subject from the acquired biorhythm of the human subject;
a controller configured to control a sound source to play a sound in accordance with the acquired biorhythm of the human subject, the sound being defined using a prescribed parameter set; and
an evaluator configured to, after awakening of the human subject,
evaluate a sleep state of the human subject for a period extending from sleep onset to the awakening, based on the estimated sleep depths of the human subject,
evaluate the parameter set based on the evaluated sleep state of the human subject, and
based on a result of evaluating the parameter set, instruct the controller to change at least a part of contents of the parameter set.

2. The sound source control device according to claim 1, wherein
in accordance with a set time point, the evaluator modifies characteristics indicative of time changes in sleep depths that are stored in advance in association with the human subject, and
the evaluator instructs the controller to change the at least a part of the contents of the parameter set such that characteristics indicative of time changes in the estimated sleep depths of the human subject approach the modified characteristics.

3. The sound source control device according to claim 2, wherein the evaluator
apportions the modified characteristics into time segments,
transmits to the controller the parameter set for each of the time segments,
instructs the controller to change the at least a part of the contents of the parameter set such that, for each of the time segments, the characteristics indicative of the time changes in the estimated sleep depths of the human subject for that segment approach the modified characteristics in that segment, and
evaluates the parameter set based on a sleep state of the human subject in each of the time segments.

4. The sound source control device according to claim 3, wherein the evaluator
transmits to the controller the parameter set to each time segment, the parameter set being one of parameter sets different from one another for respective ones of the time segments, and
evaluates each of the parameter sets based on a sleep state of the human subject in a corresponding one of the time segments.

5. The sound source control device according to claim 1, wherein
the evaluator evaluates the prescribed parameter set based on an evaluation result of the sleep, the evaluation being performed by the human subject.

6. A sound source control method, comprising:
acquiring a biorhythm of a human subject;
estimating sleep depths of the human subject from the acquired biorhythm of the human subject; and
playing a sound in accordance with the acquired biorhythm of the human subject, the sound being defined using a prescribed parameter set, and
the method further comprising:
after awakening of the human subject,
evaluating a sleep state of the human subject for a period extending from sleep onset to the awakening, based on the estimated sleep depths of the human subject;
evaluating the parameter set based on the evaluated sleep state of the human subject; and
based on a result of evaluating the parameter set, changing at least a part of contents of the parameter set.
